(19) United States
(12) Patent Application Publication
(10) Pub. No.: US 2013/0013011 A1
(43) Pub. Date: Jan. 10, 2013

(54) IMPLANTABLE MEDICAL DEVICE FOR PULSE GENERATION AND WITH MEANS FOR COLLECTING AND STORING ENERGY DURING A RECHARGE PHASE

(75) Inventors: Jorgen Edvinsson, Sollentuna (SE); Allan Olson, Spanga (SE); Marie Herstedt, Stockholm (SE); Therese Danielsson, Uppsala (SE); Ting Jun Lo, Kista (SE)

(73) Assignee: ST. JUDE MEDICAL AB, Jarfalla (SE)

(21) Appl. No.: 13/582,216

(22) PCT Filed: Mar. 31, 2010
(86) PCT No.: PCT/EP2010/054283

§ 371 Date: Aug. 31, 2012

(51) Int. Cl. A61N 1/378; A61N 1/362; A61N 1/39

(52) U.S. Cl. 607/5; 607/61; 607/33

(57) ABSTRACT

A pulse generating implantable medical device comprises a power source , a control unit, a plurality of switching units, a timing unit, a pulse generating unit adapted to generate one or more stimulation pulses to be applied to human or animal tissue via one or more stimulation electrodes, and a coupling capacitor in series with each stimulation electrode. A stimulation pulse is adapted to be applied during a stimulation pulse timing cycle that includes a stimulation phase and a recharge phase, and that the timing of a stimulation pulse timing cycle is controlled by the control unit via the timing unit and the switching units. The implantable medical device further comprises an energy storage unit and that, during the recharge phase, one or more of the switching units is adapted to establish electrical connection between the one or many stimulation electrodes and the energy storage unit in order to collect and store energy from applied stimulation pulses.

### FIELD OF THE INVENTION

The present invention relates to a pulse generating implantable medical device, and a method in relation to such a device, according to the preambles of the independent claims, and in particular to a heart stimulating device, e.g. an implantable pacemaker, cardioverter/defibrillator (ICD) or cardiac resynchronization therapy (CRT) device. The invention is also applicable in relation to other implantable stimulation devices, such as implantable nerve stimulators and general tissue stimulators.

### BACKGROUND OF THE INVENTION

One of the main design challenges with pacemaker, ICD and CRT devices today is to design a system with a longevity that is competitive and in the same time have a device capable of delivering many kinds of therapies and supporting different kinds of sensors and other functionalities like radio communications to external equipment. The only power source of today's pacemakers/ICD is the battery. The battery size is limited due to the continuous endeavour to make the device smaller. This puts a lot of pressure on making everything in a pacemaker/ICD system as power efficient as possible, which require that the electronics in the pacemaker/ICD is operated with ultra low power. One of the largest power consumers in such a device is the stimulation pulses delivered to the patient. In order to stimulate the heart muscle the stimulation pulse needs to contain a certain amount of energy. The power consumed by pacing stimulations is often more than 50% of total power utilization for pacemakers and CRTs.

The object of the present invention is to increase the longevity of an implantable medical device.

### SUMMARY OF THE INVENTION

The above-mentioned object is achieved by the present invention according to the independent claims.

Preferred embodiments are set forth in the dependent claims.

According to the present invention a pulse generating implantable medical device is provided that comprises a power source for energizing the medical device, a control unit, a plurality of switching units, a timing unit, a pulse generating unit adapted to generate one or many stimulation pulses to be applied to human or animal tissue via one or many stimulation electrodes, and a coupling capacitor in series with each stimulation electrode, wherein a stimulation pulse is adapted to be applied during a stimulation pulse timing cycle that includes a stimulation phase and a recharge phase, and that the timing of a stimulation pulse timing cycle is controlled by said control unit via said timing unit and said switching units. During the stimulation phase, said coupling capacitor(s) is/are charged, and during the recharge phase, said coupling capacitor(s) is/are discharged. The implantable medical device further comprises an energy storage unit, e.g. one or many reservoir capacitors. During the recharge phase, one or many of said switching units is adapted to establish electrical connection between said one or many stimulation electrodes and said energy storage unit in order to collect and store energy from applied stimulation pulses.

The implantable medical device and also the method used in connection with the implantable medical device are adapted to harvest (recycle) energy from the stimulation recharge phase to increase the device longevity. In one embodiment of the invention the device and method are implemented in an implantable heart stimulating device. This will increase the device longevity for all patients with a heart stimulator and especially for patients with high pacing loads, such as CRT patients, in that it makes it possible to recycle some of the pacing stimulation energy back to the pacemaker's electronic circuitry.

### SHORT DESCRIPTION OF THE APPENDED DRAWINGS

FIG. 1 is a simplified circuit diagram of a pulse generating implantable medical device according to prior art.

FIG. 2 are timing diagrams illustrating the operation of the prior art device shown in FIG. 1.

FIG. 3 is a simplified block diagram illustrating a pulse generating implantable medical device according to the present invention.

FIG. 4 is a simplified circuit diagram of a pulse generating implantable medical device according to a first embodiment according to the present invention.

FIG. 5 are timing diagrams illustrating the operation of the device shown in FIG. 4.

FIG. 6 is a simplified circuit diagram of a pulse generating implantable medical device according to a second embodiment according to the present invention.

FIG. 7 are timing diagrams illustrating the operation of the device shown in FIG. 6.

FIG. 8 is a flow diagram illustrating the method according to the present invention.

### DETAILED DESCRIPTION OF PREFERRED EMBODIMENTS OF THE INVENTION

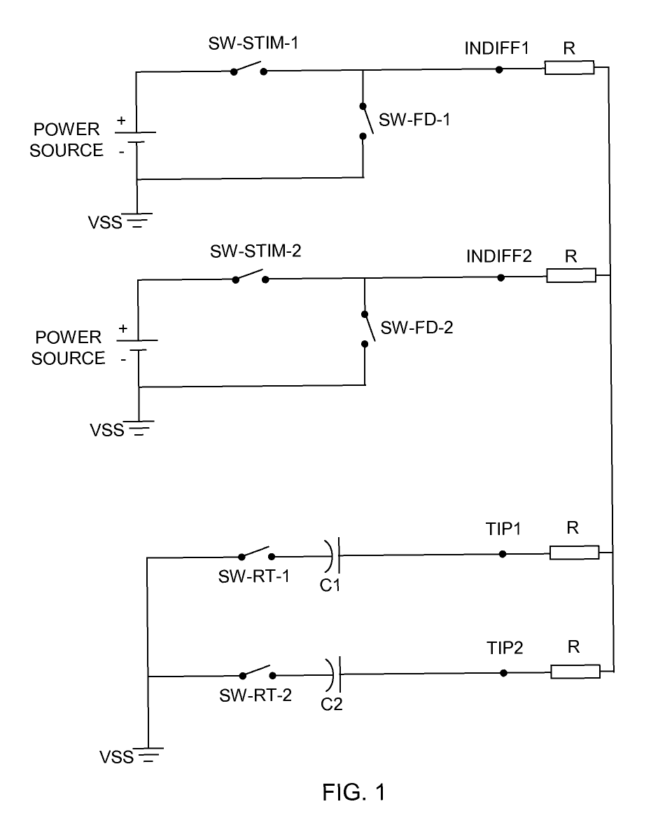

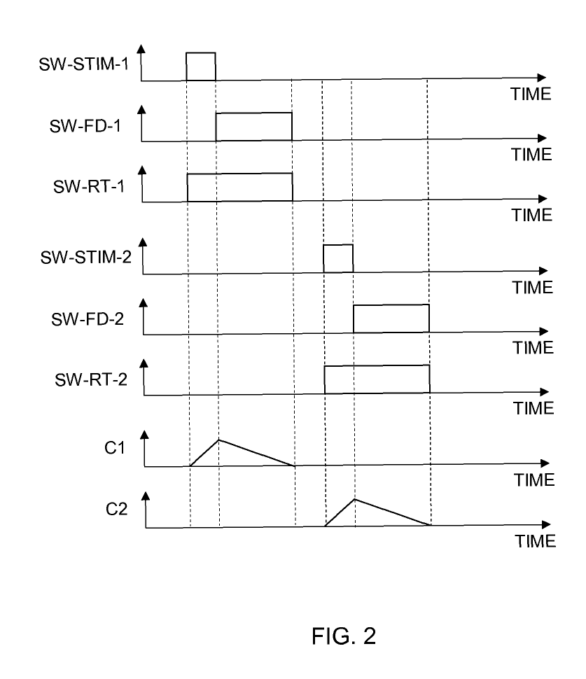

With references to FIGS. 1 and 2 the operation of a pulse generating implantable medical device according to prior art will be described in detail.

Thus, the simplified circuitry shown in FIG. 1 illustrates a conventional heart stimulator having two stimulating channels, e.g. one right atrial and one right ventricular stimulating channel.

The atrial channel comprises a stimulation tip electrode (TIP1) arranged in the atrium of the heart and used to apply stimulation pulses between the atrial tip electrode and an indifferent electrode (INDIFF1). The indifferent electrode is e.g. arranged as a ring electrode provided at the electrode lead close to the tip electrode (bipolar configuration), as an electrode at another electrode lead, or provided at the housing of the implantable device (unipolar configuration).

Similar, the ventricular channel comprises a stimulation tip electrode (TIP2) e.g. arranged in the ventricle of the heart and used to apply stimulation pulses between the ventricular tip electrode and an indifferent electrode (INDIFF2). The indifferent electrode is either arranged as a ring electrode provided at the electrode lead close to the tip electrode (bipolar configuration), as an electrode at another electrode lead, or provided at the housing of the implantable device (unipolar configuration).

For case of simplicity only one ventricular channel is illustrated in FIG. 1. The skilled person naturally realizes that further ventricular channels may be included.

Each stimulation channel consists of an anode, herein called INDIFF (indifferent electrode), and a cathode, herein called TIP (tip electrode). The stimulation sequence comprises two phases, one stimulation phase and one recharge phase.

In the stimulation phase the INDIFF potential is increased to a regulated potential relative to the TIP and a current will start flowing from INDIFF to TIP through the patient. This is achieved by closing switches SW-STIM-1 and SW-RT-1. At the TIP a coupling capacitor C1 is placed in series with the current path, this capacitor is placed inside the stimulating device/pacemaker. The current flowing through the patient during the stimulation phase charges the coupling capacitor.

Directly after the stimulation phase the recharge phase takes over.

In the recharge phase, the coupling capacitor is recharged through the patient in order to obtain a charge neutral system. The fast recharge is accomplished by connecting the

INDIFF (anode) and the cathode side of the coupling capacitor to the same node. This node is here the ground of the device called VSS. This is achieved by closing switch SW-FD-1. During this period, most of the charge is recharged. The remaining charge is slowly recharged during the following slow recharge period through a resistor (not shown in the figure) that is connected from the INDIFF to the internal side of the coupling capacitor. In the recharge phase, the same amount of charge shall be recharged through the heart electrode as in the stimulation phase. In this way, if you are measuring the charge passing through the INDIFF or TIP, there will be equal amount of charge flowing in both directions and the stimulation sequence will be charge neutral. In a heart stimulating system with more than one stimulating channel the same will be applicable for every stimulating channel. Every stimulating channel must fulfil charge neutrality and if this not is fulfilled there is a risk that the lead start to degenerate. Thus, the ventricular stimulation channel illustrated in FIG. 1 operates in a similar way as the first stimulation channel. In FIGS. 1 and 2 the switches instead is referenced as SW-STIM-2, SW-FD-2 and SW-RT-2. As shown in FIG. 2 the second channel is activated after the first channel. The voltage over the second coupling capacitor C2 is also illustrated in FIG. 2.

In a typical application in a heart stimulating device the switches SW-STIM-1/2 are closed approximately 0.5-1.5 ms and switches SW-RT-1/2 are closed approximately 8-12 ms. However, these time periods may naturally have other durations.

Thus, essentially all the energy in the stimulation pulse that has stimulated the heart muscle is dumped in the recharge phase.

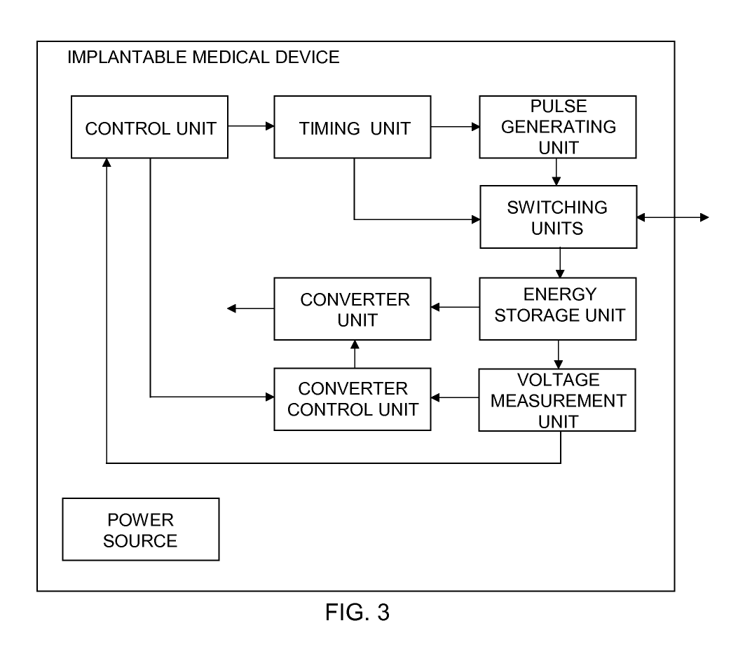

FIG. 3 is a simplified block diagram illustrating a pulse generating implantable medical device according to the present invention.

The block diagram in FIG. 3 is not exhaustive and further functional units are naturally included, e.g. communication units, a storage unit, and sensors units, but are omitted in that those units are not directly related to the present invention.

With references to FIG. 3, the present invention relates to a pulse generating implantable medical device comprising a power source for energizing the medical device, a control unit, a plurality of switching units controlled by the control unit, a timing unit and a pulse generating unit adapted to generate one or many stimulation pulses to be applied to human or animal tissue via one or many stimulation electrodes.

The control unit controls the timing unit that in turn controls the pulse generating unit and switching unit to generate the stimulation pulses, e.g. in dependence of one or many stimulation modes implemented by the control unit. In the figure the double-arrow from the switching unit refers both to one or many stimulation channels, e.g. atrial electrode leads, right ventricular electrode leads, left ventricular electrode leads, nerve stimulating electrode leads, etc., each provided with one or many stimulation electrodes, and also to detected sensor signals, e.g. electrical tissue responses, depolarization responses.

A stimulation pulse is adapted to be applied during a stimulation pulse timing cycle that includes a stimulation phase and a recharge phase, and that the timing of a stimulation pulse timing cycle is controlled by the control unit via the timing unit and the switching units.

The implantable medical device further comprises an energy storage unit. During the recharge phase, one or many of the switching units is/are adapted to establish electrical connection between the one or many stimulation electrodes and the energy storage unit in order to collect and store energy from applied stimulation pulses.

In one embodiment the energy storage unit comprises one or many reservoir capacitors, generally designated C_{R}.

If the energy storage unit comprises at least two reservoir capacitors those may be arranged in parallel and being independently connectable by the control unit to achieve a variable capacitance of the energy storage unit.

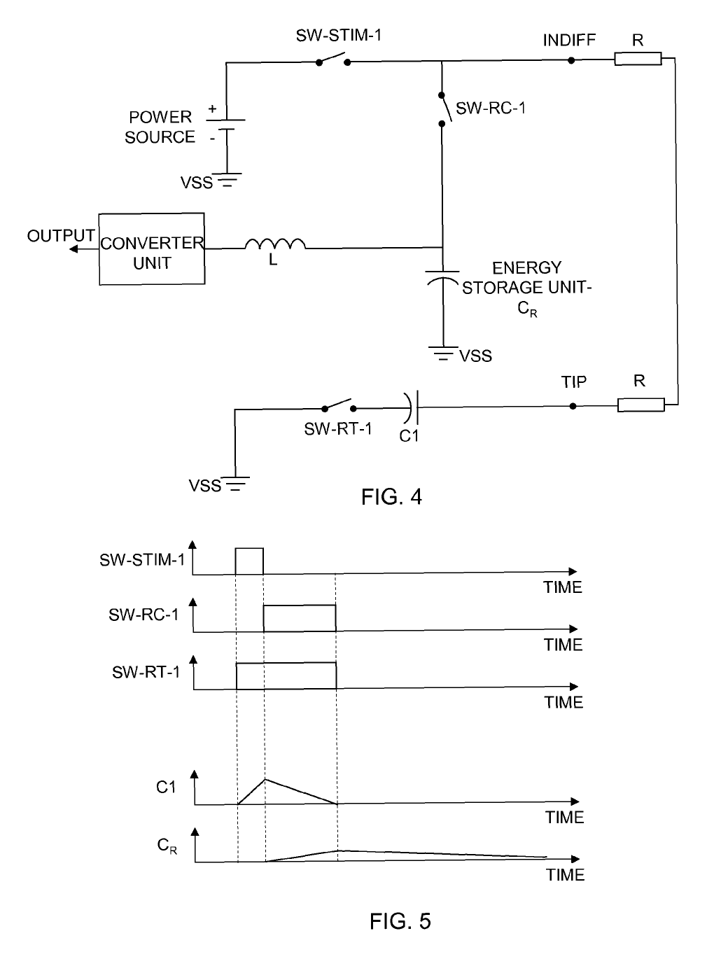

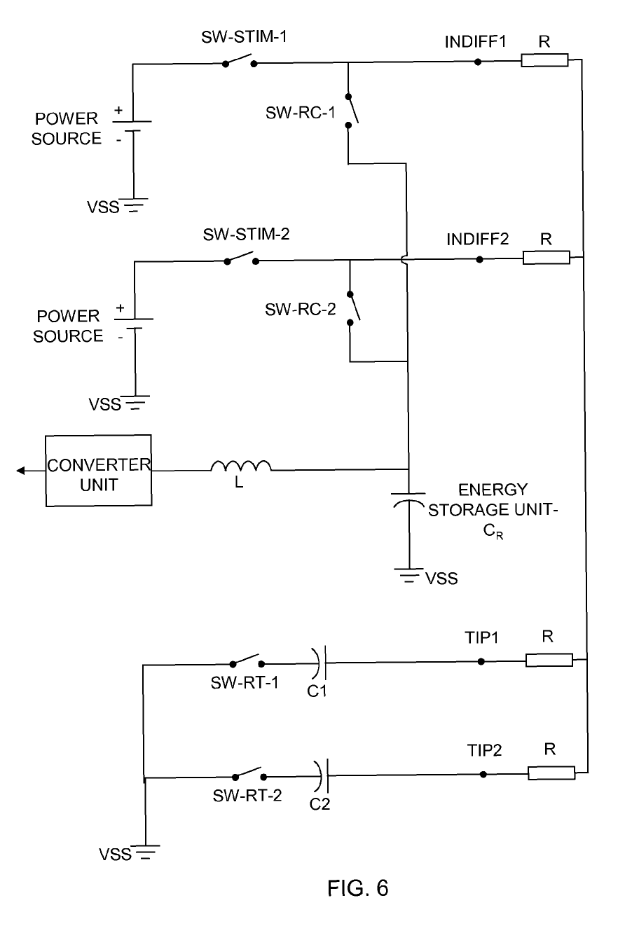

The capacitance of the one or many reservoir capacitors is preferably related to the coupling capacitors of the medical device (see FIGS. 4 and 6) and is approximately at least three times the capacitance of one coupling capacitor. In one typical example the energy storage unit may have a capacitance of at least 50 μF.

Another issue that influences the capacitance of the energy storage unit is the relation to the operating voltage of the medical device which is often approximately 2 Volts.

Taken that into account, the capacitance must be sufficiently high such that the voltage over the capacitor does not exceed a predetermined level, e.g. 0.3 V.

FIG. 4 is a simplified circuit diagram of a pulse generating implantable medical device according to a first embodiment according to the present invention. In this embodiment the medical device comprises one stimulation channel.

FIG. 5 are timing diagrams illustrating the operation of the device shown in FIG. 4. The purpose of FIG. 5 is to illustrate the principle of the operation, e.g. the timing of opening and closing of the switches.

The stimulation channel comprises a stimulation tip electrode (TIP1), e.g. adapted to be arranged in the atrium or the ventricle of the heart, and used to apply stimulation pulses between the tip electrode and an indifferent electrode (INDIFF1). The indifferent electrode is either arranged as a ring electrode provided at the electrode lead close to the tip electrode (bipolar configuration) or provided at the housing of the implantable device (unipolar configuration).

In the stimulation phase the INDIFF potential is increased to a regulated potential relative to the TIP and a current will start to flow from INDIFF to TIP through the patient. This is achieved by closing switches SW-STIM-1 and SW-RT-1. At the TIP a coupling capacitor C1 is placed in series with the current path, this capacitor is placed inside the stimulating device/pacemaker. The current flowing through the patient during the stimulation phase charges the coupling capacitor.

After a predetermined time period, e.g. 0.5-1.5 ms, switch SW-STIM-1 is opened and thereafter a switch SW-RC-1 is closed. The switch SW-RC-1 is arranged such that it connects the INDIFF pole to the energy storage unit when it is closed. The energy storage unit is connected to switch SW-RC-1 and to the device ground VCC. The converter unit is connected to the energy storage unit via an inductor L and is adapted to generate a converter output voltage signal.

The switch SW-RT-1 at the TIP pole and the switch SW-RC-1 that connects the INDIFF pole to the energy storage unit remain closed for a predetermined time period, e.g. 8-15 ms. During that time period the voltage over the energy storage unit increases up to a level related to the capacitance of the energy storage unit. After that the energy storage unit is slowly discharged by the converter unit. This is illustrated by the last diagram in FIG. 5.

According to another embodiment an additional inductor is included between the switch SW-RC-1 and the energy storage unit for the reason to filter the current peaks which may be present, this will result in a higher efficiency.

In one embodiment the device further comprises a voltage measurement unit adapted to measure the voltage over the energy storage unit and to generate a measurement signal in dependence thereto. That signal is applied to the control unit and preferably also to a converter control unit. The generated stimulation pulse amplitudes may be adjusted in dependence of the measurement signal, i.e. the voltage over the energy storage unit.

The voltage over the energy storage unit, e.g. the reservoir capacitor, when a stimulation pulse starts and the capacitance relation between the coupling capacitor and the reservoir capacitor will set the limit on how low the voltage over the coupling capacitor can be discharged to during the fast discharge period. If there is a voltage left on the coupling capacitor until the next stimulation pulse the amplitude of that stimulation pulse will have to be decreased equally much. The voltage on the reservoir capacitor can momentarily be high as long as the voltage level is low before a new stimulation pulse is to begin.

The energy storage unit is connected to a converter unit adapted to convert stored energy at the energy storage unit to a voltage level applicable for the medical device. The converter unit is controlled by a converter control unit such that the voltage level is adapted to one of several stimulation modes applied by said medical device. The converter unit may also be controlled by the converter control unit such that the voltage level approximately corresponds to the supply voltage level of the medical device, e.g. in the interval 1.8-4.0 V.

In one embodiment the converter unit is a DC-DC converter where the low voltage 4.2-0.3V is up-converted to a voltage in the range of the supply voltage in the range of 1.8-4.0V. In this way, the stimulating energy is recycled and can be used by the pacemaker's electronic circuitry again. The DC-DC converter is controlled by the converter control unit in order to achieve highest possible efficiency for different operating modes.

In one embodiment the converter control unit is controlled such that an operating mode for CRT pacing with high energy stimulation pulses is achieved and in another embodiment the converter control unit is controlled such that an operating mode for dual chamber pacing with lower energy stimulation pulses is achieved.

The control unit may regulate the switching duty cycle and also control when it shall be turned on and off depending on both the voltage on the reservoir capacitor and on the output voltage of the DC-DC converter.

The converter unit shall be able to adapt the way it converts the voltage from the reservoir capacitor to a supply voltage depending on the charge rate (charge/cardiac cycle) stored on the reservoir capacitor, i.e. high power stimulation or low power stimulation, and the output voltage from the converter unit.

Furthermore, the converter unit shall be able to be programmed to a desired output voltage in the range of 1.8 to 4.0V.

FIG. 6 is a simplified circuit diagram of a pulse generating implantable medical device according to a second embodiment according to the present invention.

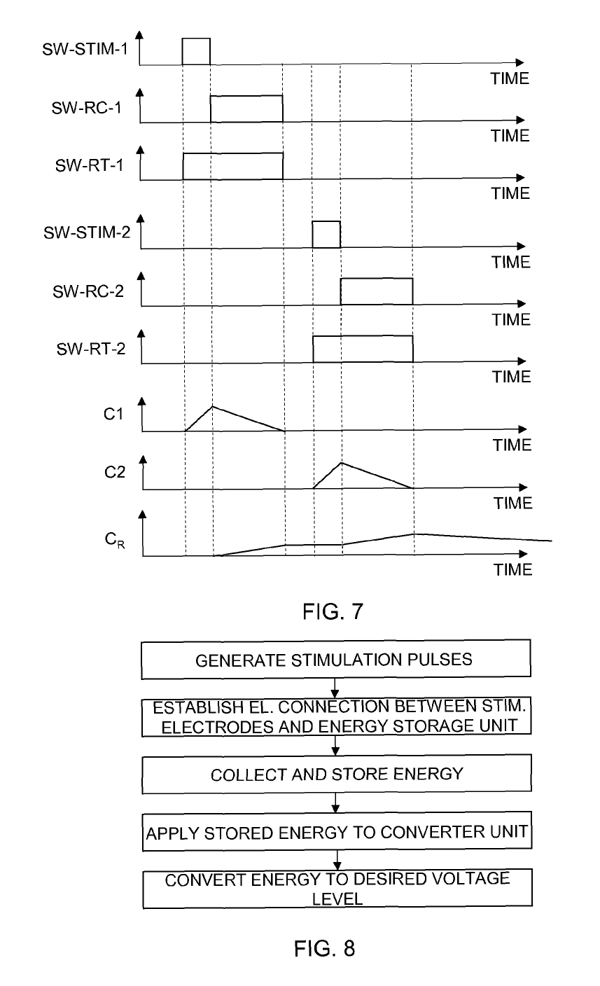

FIG. 7 are timing diagrams illustrating the operation of the device shown in FIG. 6. In the second embodiment the medical device comprises two stimulation channels, e.g. one right atrial and one right ventricular stimulating channel.

The atrial channel comprises a stimulation tip electrode (TIP1) arranged in the atrium of the heart and used to apply stimulation pulses between the atrial tip electrode and an indifferent electrode (INDIFF1). The indifferent electrode is either arranged as a ring electrode provided at the electrode lead close to the tip electrode (bipolar configuration) or provided at the housing of the implantable device (unipolar configuration).

Similar, the ventricular channel comprises a stimulation tip electrode (TIP2) arranged in the ventricle of the heart and used to apply stimulation pulses between the ventricular tip electrode and an indifferent electrode (INDIFF2). The indifferent electrode is either arranged as a ring electrode provided at the electrode lead close to the tip electrode (bipolar configuration) or provided at the housing of the implantable device (unipolar configuration).

For case of simplicity only one ventricular channel is illustrated in FIG. 6. The skilled person naturally realizes that further ventricular channels may be included. For example a left ventricular channel provided with a tip electrode using one of the two indifferent electrodes as counter electrode.

As mentioned above the stimulation sequence comprises two phases, one stimulation phase and one recharge phase, where the recharge phase is typically divided into a fast recharge period and a slow recharge period.

In the stimulation phase of the first channel the INDIFF1 potential is increased to a regulated potential relative to the TIP1 and a current will start to flow from INDIFF1 to TIP1 through the patient. This is achieved by closing switches SW-STIM-1 and SW-RT-1. At TIP1 a coupling capacitor C1 is placed in series with the current path, this capacitor is placed inside the stimulating device/pacemaker. The current flowing through the patient during the stimulation phase charges the coupling capacitor C1.

In the stimulation phase of the second channel the INDIFF2 potential is increased to a regulated potential relative to the TIP2 and a current will start to flow from INDIFF2 to TIP2 through the patient. This is achieved by closing switches SW-STIM-2 and SW-RT-2. At TIP2 a coupling capacitor C2 is placed in series with the current path, this capacitor is placed inside the stimulating device/pacemaker. The current flowing through the patient during the stimulation phase charges the coupling capacitor C2.

Directly after the stimulation phase the recharge phase takes over.

In a typical application in a heart stimulating device the switches SW-STIM-1/2 are closed approximately 0.5-1.5 ms and switches SW-RT-1/2 are closed approximately 8-15 ms. However, these time periods may naturally have other durations.

After a predetermined time period, e.g. 0.5-1.5 ms, switch SW-STIM-1 is opened and thereafter a switch SW-RC-1 is closed. The switch SW-RC-1 is arranged such that it connects the INDIFF1 pole to the energy storage unit when it is closed. The energy storage unit is connected to switch SW-RC-1 and to the device ground VCC. The converter unit is connected to the energy storage unit via an inductor L and is adapted to generate a converter output voltage signal.

The switch SW-RT-1 at the TIP1 pole and the switch SW-RC-1 that connects the INDIFF1 pole to the energy storage unit remain closed for a predetermined time period, e.g. 8-15 ms. During that time period the voltage over the energy storage unit increases up to a level related to the capacitance of the energy storage unit.

A similar operation as described above in relation to the first stimulation channel is performed by the second stimulation channel. Thus, the second stimulation channel illustrated in FIG. 6 operates in a similar way as the first stimulation channel. In FIGS. 6 and 7 the switches instead are referenced to as SW-STIM-2, SW-RC-2 and SW-RT-2. As shown in FIG. 7 the second channel is activated after the first channel. The voltage over the second coupling capacitor C2 is also illustrated in FIG. 7.

The voltage over the energy storage unit after the second stimulation channel has ended is essentially the sum of the voltages from C1 and C2. This is illustrated by the last diagram in FIG. 7. After that the energy storage unit is slowly recharged by the converter unit.

The second embodiment, described with references to FIGS. 6 and 7, thus is provided with a converter unit. All features described in relation to the first embodiment in relation to the converter unit, the energy storage unit, the converter control unit and the voltage measurement unit are naturally also applicable for this second embodiment.

Thus, the medical device according to the present invention, and with references to FIG. 4 or 6, is provided with one or many stimulation channels, and for each stimulation channel a stimulation pulse is adapted to be applied between a first electrical pole (INDIFF, INDIFF1, INDIFF2) and a second electrical pole (TIP, TIP1, TIP2), where each of said poles is connectable to a respective stimulation electrode. The first pole is connectable to a positive pole of the power source via a first switching unit (SW-STIM-1, SW-STIM-2) and is also connectable to said energy storage unit via a second switching unit (SW-RC-1, SW-RC-2). The second pole is connectable to device ground (VSS) via a third switching unit (SW-RT-1, SW-RT-2) and a capacitor (C1, C2).

The one or many stimulation electrodes is/are arranged at the implantable medical device, or at one or many electrode leads connectable to said implantable medical device.

The present invention also relates to a method in a pulse generating implantable medical device, the method comprises:
a) generating stimulation pulses to be applied to human or animal tissue via one or many stimulation electrodes, a stimulation pulse having a stimulation pulse timing cycle including a stimulation phase and a recharge phase;
b) switching, during said recharge phase, a switching unit to establish an electrical connection between said one or many stimulation electrodes and an energy storage unit, and
c) collecting and storing energy from applied stimulation pulses at said energy storage unit.

Furthermore, the method comprises, after c),
d) applying the energy stored at said energy storage unit to a converter unit,
e) converting energy received by said converter unit to a voltage level applicable for the medical device.

The method is illustrated by the flow diagram in FIG. 8.

In the following the size of the reservoir capacitor (energy storage unit) is discussed, in particular the relation between coupling capacitor size and reservoir capacitor size.

The following formula describes the coupling capacitor voltage limit after a discharge depending on initial voltage on capacitors and the size of the capacitors:

$${u}_{C} = {u}_{C} ( 0 ) \frac{{C}_{C}}{{C}_{C} + {C}_{R}} + {u}_{R} ( 0 ) \frac{{C}_{R}}{{C}_{C} + {C}_{R}}$$

where,
u_{C}(0)=initial voltage on coupling capacitor
   u_{R}(0)=initial voltage on reservoir capacitor

If calculating on a typical case in high power stimulation mode. Stimulation pulse amplitude=7V, pulse width=1 ms, load=500 ohm this will give a u_{C}(0)=2.7V assume u_{R}(0)=0V and the goal is to bring down the voltage on the coupling capacitor to <0.2V.

$$⇒ \frac{{C}_{R}}{{C}_{C}} = \frac{{u}_{C} ( 0 ) - {u}_{C}}{{u}_{C}} = \frac{2.7 - 0.2}{0.2} = 12.5$$

The same calculation in a normal power stimulation mode, stimulation pulse=3V, pulse width=0.5 ms, load=500 ohm. This will give a u_{C}(0)=0.93V. With the same goal of bringing down the voltage on the coupling capacitor to <0.2V will lead to

$$⇒ \frac{{C}_{R}}{{C}_{C}} = 3.6$$

The invention will be most efficient when we have a low pacing efficiency which is the case when stimulating in a high power mode (when we have large amount of charge transferred in stimulation phase). The coupling capacitor will capture a bigger part of the energy which can be reused by the invention.

The reservoir capacitance would with these different possible stimulation modes be adjustable to adapt to the stimulation mode. A low capacitance mode and a high capacitance mode which will be used only during high power stimulation episodes. As discussed above the reservoir capacitor could be two capacitors which will give three different capacitance levels. Or the capacitor could also be only one with a capacitance most suited for the high power stimulation modes. The low power stimulation mode might not gain any energy to recycle.

A first option is to have one reservoir capacitor which is ˜10 times bigger than the coupling capacitor. Second option is to have two reservoir capacitor that can be individually connected (let say we had CR1=20 uF, CR2=40 uF then we have the ability to get 20 uF, 40 uF and 60 uF).

The present invention is not limited to the above-described preferred embodiments. Various alternatives, modifications and equivalents may be used. Therefore, the above embodiments should not be taken as limiting the scope of the invention, which is defined by the appending claims.

1. A pulse generating implantable medical device comprising:
a power source energizing the medical device;
a control unit;
a plurality of switching units;
a timing unit;
a pulse generating unit adapted to generate stimulation pulses to be applied to human or animal tissue via one or more stimulation electrodes;
a coupling capacitor in series with each stimulation electrode, wherein a stimulation pulse is adapted to be applied during a stimulation pulse timing cycle that includes a stimulation phase and a recharge phase, and that the timing of a stimulation pulse timing cycle is controlled by the control unit via the timing unit and the switching units; and
an energy storage unit and that one or more of the switching units is adapted to establish, during the recharge phase, electrical connection between the one or more stimulation electrodes and the energy storage unit in order to collect and store energy from applied stimulation pulses.

2. The implantable medical device according to claim 1, wherein the energy storage unit comprises one or more reservoir capacitors.

3. The Implantable medical device according to claim 2, wherein the capacitance of the one or more reservoir capacitors is at least three times the capacitance of the coupling capacitor.

4. The Implantable medical device according to claim 1, wherein the energy storage unit comprises at least two reservoir capacitors arranged in parallel and being independently connectable by said the control unit to achieve a variable capacitance of the energy storage unit.

5. The Implantable medical device according to claim 1, wherein the energy storage unit has a capacitance of at least 50 μF.

6. The Implantable medical device according to claim 1, wherein the energy storage unit has a capacitance that is sufficiently high such that the voltage over the capacitor does not exceed a predetermined level, e.g. 0.3 V.

7. The Implantable medical device according to claim 1, wherein the device further comprises a voltage measurement unit adapted to measure the voltage over the energy storage unit and to generate a measurement signal in dependence thereto that is applied to the control unit.

8. The Implantable medical device according to claim 7, wherein generated stimulation pulse amplitudes are adjusted in dependence of the measurement signal.

9. The Implantable medical device according to claim 1, wherein the energy storage unit is connected to a converter unit adapted to convert stored energy to a voltage level applicable for the medical device.

10. The Implantable medical device according to claim 9, wherein the converter unit is controlled by a converter control unit such that the voltage level is adapted to one of several stimulation modes applied by the medical device.

11. The Implantable medical device according to claim 9, wherein the converter unit is controlled by a converter control unit such that the voltage level approximately corresponds to the supply voltage level of the medical device, e.g. in the interval 1.8-4.0 V.

12. The Implantable medical device according to claim 1, wherein the medical device is provided with one or more stimulation channels, and for each stimulation channel a stimulation pulse is adapted to be applied between a first electrical pole (INDIFF, INDIFF1, INDIFF2) and a second electrical pole (TIP, TIP1, TIP2), wherein each of said the poles is connectable to a respective stimulation electrode, wherein the first pole is connectable to a positive pole of the power source via a first switching unit (SW-STIM-1, SW-STIM-2) and is also connectable to the energy storage unit via a second switching unit (SW-RC-1, SW-RC-2), and wherein the second pole is connectable to device ground (VSS) via a third switching unit (SW-RT-1, SW-RT-2) and a capacitor (C1, C2).

13. The Implantable medical device according to claim 1, wherein said the one or many electrodes is/are arranged at the implantable medical device, or at one or more electrode leads connectable to the implantable medical device.

14. The Implantable medical device according to claim 1, wherein the device is an implantable heart stimulator.

15. A method in a pulse generating implantable medical device comprising:
a) generating stimulation pulses to be applied to human or animal tissue via one or more stimulation electrodes, a stimulation pulse having a stimulation pulse timing cycle including a stimulation phase and a recharge phase;
b) switching, during the recharge phase, a switching unit to establish an electrical connection between the one or more stimulation electrodes and an energy storage unit; and
c) collecting and storing energy from applied stimulation pulses at the energy storage unit.

16. Method The method according to claim 15, further comprising after the collecting and storing energy:
d) applying the energy stored at the energy storage unit to a converter unit; and
e) converting energy received by the converter unit to a voltage level applicable for the medical device.
